(19) United States
(12) Patent Application Publication
(10) Pub. No.: US 2004/0037786 A1
(43) Pub. Date: Feb. 26, 2004

(54) Aerosol foam product for hair treatment

(76) Inventors: Susanne Birkel, Darmstadt (DE); Harald Wendel, Ober-Ramstadt (DE); Michael Franzke, Rossdorf (DE); Hildegard Henze, Darmstadt (DE); Manuela Hannich, Egelsbach (DE)

Correspondence Address: STRIKER, STRIKER & STENBY, 103 East Neck Road, Huntington, NY, 11743, US

(21) Appl. No.: 10/606,046

(22) Filed: Jun. 25, 2003

(30) Foreign Application Priority Data
Jun. 26, 2002 (DE) 102 28 436.9

(51) Int. Cl. A61K 9/00

(52) U.S. Cl. 424/47

(57) ABSTRACT

The aerosol foam product for hair treatment includes a pressure-resistant container and a foamable composition filled in the pressure-resistant container together with a propellant. The foamable composition contains a dialkyldiallyl ammonium chloride/acrylamide copolymer; another copolymer, formed from a first monomer, which is an alkyl acrylate or alkylmethacrylate, and a second monomer, which is acrylic acid or methacrylic acid; at least one base for at least partial or total neutralization of the another copolymer and a suitable solvent system.

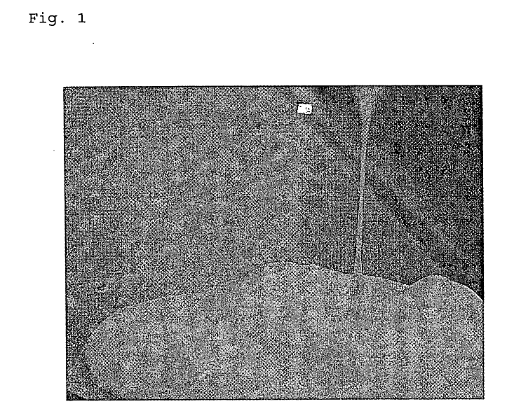

### BACKGROUND OF THE INVENTION

1. Field of the Invention

The subject matter of the invention is an aerosol foam product for hair treatment including a composition containing a dialkyldiallyl ammonium chloride/acrylamide copolymer and an at least partially or complete neutralized copolymer of an alkyl acrylate or methacrylate and acrylic or methacrylic acid.

2. Description of the Related Art

Foams for hair treatment are known and usually include hair-fixing or hair-care substances, foam-formers and a suitable aqueous solvent base. These compositions are foamed prior to application by means of a propellant gas or by means of a mechanically operated pump foam apparatus. These different types of foam products have different requirements regarding their quality, which can be roughly divided into two categories or groups. The first group of quality requirements deals with foam quality, i.e. the properties of the foam. These foam properties include fine porosity or coarse porosity, compactness, stability of the foam over time, workability into the hair and distributability of it on the hair. The second group of quality requirements relate to the action, which is produced by the foam after working it into the hair, i.e. the hair-care properties, such as the feel of the hair under moist and dry conditions, the combability, the fixing action, the load on the hair, the luster of the hair, etc.

The problem with the optimization of the foam products is that improvements of some quality requirements, for example the foam quality, can be achieved by addition of special active ingredients. However this usually results in the impairment of other quality requirements, for example the hair care or hair fixing properties. Frequently cationic polymers are contained in foam products because of their good substantive activity on human hair.

Aerosol foams are known from DE 32 17 059, which contain a combination of a certain cationic polymer and anionic polymer. Non-aerosol pump foams are known from WO 99/25311, which contain a combination of certain cationic and anionic polymers.

### SUMMARY OF THE INVENTION

It is an object of the present invention to further improve and optimize the quality and specifications of foam products for treating hair, especially the foam quality and hair care and/or hair fixing properties of these foam produced.

It has now been found that this object is attained by the foam product described in the following description and claimed in the following claims.

The term “aerosol foam product” understandably means a product, which comprises a liquid foamable composition, which is filled into a pressurized or pressure-resistant container with a foam head together with a propellant gas. Foam is produced from the liquid foamable composition immediately prior to application by using the foam head.

The subject matter of the invention is an aerosol foam product for hair treatment, which includes a composition containing

(A) at least one dialkyldiallyl ammonium chloride/acrylamide copolymer;

(B) at least one other copolymer, formed from at least one first monomer and at least one second monomer, the first monomer being an alkyl acrylate and/or alkylmethacrylate and the at least one second monomer being acrylic acid and/or methacrylic acid;

(C) at least one base for at least partial or total neutralization of the at least one other copolymer (B), and

(D) a suitable solvent system;

wherein the composition is filled in a pressure-resistant container with at least one propellant (E).

The copolymer (A) is contained in the composition according to the invention preferably in an amount of 0.01 to 10, especially preferably from 0.1 to 5, percent by weight. The copolymer (B) is contained in the compositions according to the invention preferably in an amount of 0.1 to 10, especially preferably from 0.5 to 5, percent by weight. The weight ratio of the copolymer (B) to copolymer (A) is preferably in a range of greater than 1:1 to less than 10:1, especially preferably in a range of 2:1 to 5:1, The ratio of the negative charge on the copolymer (B) to the positive change on the copolymer (A) is preferably in a range of 1:1 to 10:1, especially preferably 3:1 to 8:1.

In an especially preferred embodiment the foam formed by foaming the composition according to the invention has strand drawing or forming properties. The subject matter of the invention is an aerosol foam product for hair treatment, comprising a foamable composition containing at least one hair-fixing polymer, at least one propellant and a suitable solvent system. This foamable composition is filled in a pressurized or pressure-resistant container. The foam formed from the foamable composition by dispensing it from the container has strand or fiber drawing properties. Strand drawing or forming properties are detected when a web or strand of fresh foam of a diameter of about 2 mm, preferably less than 1 mm, is formed between two fingers, e.g. the thumb and the index finger, when the foam is taken between the fingers and the fingers are moved apart from each other. Strands or webs of foam can also be detected when the foam is applied to a base, the foam surface is contacted with a finger and the finger is drawn away from the foam. The very good application properties of the foam after working into the hair probably can be ascribed to the web or strand drawing property and the improved wetting of the hair.

When applied to human hair for hairstyling the foam product according to the invention is characterized by good moist combability, good styling properties, good shaping, good curl definition, good feel, especially a smooth feel, and a reduced load. Good styling properties means that that the hair is easily brushed with a hair brush during the hairstyling when moist hair is treated with the foam product according to the invention.

Because of the good curl definition the composition according to the invention is especially suited for treating curly or waved hair. The subject matter of the invention also includes a method of using a composition for treating or preparing curled or waved hair (curl definition). This composition contains (A) at least one dialkyldiallyl ammonium chloride/acrylamide copolymer; (B) at least one other copolymer, formed from a first monomer and at least one second monomer, the first monomer being an alkyl acrylate or alkylmethacrylate and the at least one second monomer being an acrylic acid and/or methacrylic acid; (C) at least one base for at least partial or total neutralization of the at least one other copolymer (B), and (D) a suitable solvent system;

Dialkyldiallyl Ammonium Chloride/Acrylamide Copolymer (A)

The alkyl groups of the dialkyldiallyl ammonium chloride/acrylamide copolymer (A) have preferably one to four carbon atoms. Suitable dialkyldiallyl ammonium chloride/acrylamide copolymers have the INCI name Polyquaternium-7 and are marketed under the trademark MERQUAT®. Polymers with a molecular weight in a range from 500,000 to 20,000,000 g/mol, especially from 1,000,000 to 10,000,000 g/mol, are preferred. Preferred cationic charge densities of these polymers, expressed as molecular weight per unit cationic charge, are in a range from 200 to 250. For example, MERQUAT® 550 or MERQUAT® 550 L, are suitable.

Alkyl(Meth)Acrylate/(Meth)Acrylic Acid Copolymer (B)

Copolymer (B) is formed from at least one first monomer type, which is selected from the group consisting of alkyl acrylates and alkylmethacrylates, and at least one second monomer type, which is selected from the group consisting of acrylic acid and methacrylic acid. The alkyl groups preferably have one to four carbon atoms, e.g. a methyl, ethyl, propyl, isopropyl or butyl group. Preferred copolymers include alkylacrylate/methylacrylic acid copolymers. For example, ethylacrylate/methacrylic acid copolymer (INCI-name acrylates copolymer) is suitable. This latter copolymer is sold under the trademark LUVIFLEX® SOFT.

Neutralization Agent (C)

The copolymer (B) is present in completely or partially neutralized form. The neutralization degree is preferably at least 50%, especially preferably from 70 to 100%, based on the number of free acid groups. In principle all cosmetically compatible inorganic or organic bases can be used. For example, aminoalkanols, such as aminomethylpropanol (AMP), triethanolamine or monoethanol amine, but also ammonia, NaOH, KOH, among others, can be used. Among the aminoalkanols, AMP is particularly preferred.

Solvent System (D)

The composition according to the invention is embodied in a purely aqueous or in an aqueous-alcoholic medium with preferably at least 50 percent by weight water and at least 5 percent by weight alcohol. The composition according to the invention preferably contains from 50 to 98, especially preferably from 75 to 95, percent by weight of the solvent system. The alcohol particularly can be a lower alcohol with one to four carbon atoms that is usually used for cosmetic purposes, e.g. ethanol and isopropanol. Organic solvents or mixtures of solvents with boiling points under 400° C. can be used as co-solvents in an amount of 0.1 to 15, preferably 1 to 10, percent by weight. Branched or unbranched hydrocarbons, such as pentane, hexane and isopentane, and cyclic hydrocarbons, such as cyclohexane and cyclopentane, are suitable as co-solvents. Glycerol, ethylene glycol and propylene glycol are especially preferred as water-soluble solvents.

Propellant (E)

The propellant (E) is preferably contained in the aerosol foam according to the invention in an amount of 1 to 20, especially preferably from 2 to 10, percent by weight. For example, lower alkanes, such as n-butane, i-butane, propane, butane or their mixtures, dimethyl ether or fluorinated hydrocarbons, such as 1,1-difluoroethane or tetrafluoroethane or their mixtures, or pressurized gaseous propellants, e.g. N₂, N₂O and CO₂ and their mixtures, are suitable as the propellant in the composition according to the invention. Hydrocarbons with three to four carbon atoms are especially preferred as the propellant.

Surfactant (F)

A preferred embodiment of the foam product according to the invention contains at least one surfactant (F). The surfactant (F) is preferably contained in an amount of 0.01 to 15, especially preferably from 0.05 to 10, percent by weight. Suitable surfactants have emulsifying, solvating, foam-forming, foam-strengthening or hair-care properties, are preferably cationic or nonionic and have an HLB value of at most 20, preferably from 5 to 18. Especially preferred embodiments according to the invention contain both a nonionic surfactant and a cationic surfactant.

Ethoxylated surfactants, in which the ethylene oxide units are between 1 to 1000, preferably from 1 to 300, especially preferably from 1 to 15, are preferred nonionic surfactants. Fatty acid glyceride ethoxylates, fatty alcohol ethoxylates, fatty amine ethoxylates, fatty acid alkanol amide ethoxylates and fatty acid ester ethoxylates with one to fifty ethylene oxide units respectively are preferred. Suitable fatty alcohol ethoxylates are, for example, ethoxylated lauryl, tetradecyl, cetyl, oleyl or stearyl alcohol, which can be used alone or in a mixture, as well as fatty alcohols of ethoxylated lanolin or ethoxylates of lanolin. Also ethoxylated fatty alcohols, which are marked under the trademark DEHYDOL® of Henkel or under the trademark BRIJ® of ICI Surfactants, are suitable for hair treatment compositions according to the invention. Under the fatty acid ester ethoxylates, above all, diglyceride ethoxylates, should be named, for example ethoxylated castor oil with 25 ethylene oxide units with the INCI name PEG-25 hydrogenated castor oil (ARLATONE® G), ethoxylated castor oil with 35 ethylene oxide units with the INCI name PEG-35 castor oil (CREMOPHOR® E1) or ethoxylated hydrogenated castor oil ethoxylated with 40 ethylene oxide units with the INCI name PEG-40 hydrogenated castor oil (CREMOPHOR® RH 410). Additional suitable nonionic surfactants include ethoxylated fatty acid sugar esters, especially ethoxylated sorbitan fatty acid esters, which are known as polysorbates, but also non-ethoxylated surfactants, such as fatty acid sugar esters, which are marketed under the trademark TWEEN® and ARLACEL®) by ICI Surfactants and alkylpolyglycosides, which are marked under the trademark PLANTAREN® or PLANTACARE® by Henkel or under the trademark ORAMIX® by Seppic.

Suitable cationic surfactants have a quaternary ammonium group and can be represented by the general formula (I):

N⁽⁺⁾R¹R²R³R⁴X⁽⁻⁾ (I),

wherein R¹, R², R³ and R⁴, independently of each other, each represent an alkyl group, an aryl group, an alkoxy group, a polyoxyalkylene group, an alkylamido group, a hydroxyalkyl group, an aryl group or an alkyaryl group with 1 to 22 carbon atoms and X⁽⁻⁾ represents an anion, e.g. a halogen, an acetate, phosphate, nitrate or alkyl sulfate, preferably a chloride or bromide. The aliphatic groups can also contain cross-bonds or other groups, such as amino groups, in addition to carbon and hydrogen. For example, suitable cationic surfactants are the chlorides or bromides of alkyldimethylbenzyl ammonium salts or alkyl-trimethyl ammonium salts, e.g. cetyltrimethylammonium chloride or -bromide, tetradecyltrimethylammonium chloride or -bromide, alkyldimethylhydroxyethylammonium chloride or -bromide, dialkyldimethylammonium chloride or -bromide, alkylpyridinium salts, e.g. lauryl- or cetyl-pyridinium chloride, alkylamidoethyltrimethylammonium ether sulfate and compounds with a cationic character, such as amine oxides, e.g. alkyl-methylamine oxides or alkylaminoethyldimethylamine oxides. Cetyltrimethylammonium chloride is especially preferred.

The product according to the invention also contains at least one film-forming nonionic polymer in a further preferred embodiment. The nonionic polymer is preferably contained in an amount of 0.01 to 15 percent by weight, especially preferably from 0.5 to 10 percent by weight. It can be a synthetic, natural or a modified natural polymer. Especially those polymers, which have a sufficient solubility in water or water/alcohol mixtures, so that they can be present in a completely dissolved form in the compositions according to the invention, are preferred. The term “film-forming polymers” means those polymers, which when used in an 0.01 to 5 percent aqueous, alcoholic or aqueous-alcoholic solution or dispersion are in a position, to deposit a polymer film on the hair.

Suitable synthetic nonionic film-forming polymers include homopolymers or copolymers, which are built up from at least one of the following monomers: vinyl pyrrolidone, vinyl caprolactam, vinyl ester (e.g. vinyl acetate), vinyl alcohol, acrylamide, methacrylamide, alkylacrylamides, dialkylacrylamides, alkylmethacrylamides, dialkylmethacrylamides, alkylacrylates, alkylmethacrylates, propylene glycol or ethylene glycol, in which the alkyl groups of these monomers are preferably C1- to C7-alkyl groups, especially preferably C1- to C3-alkyl groups. For example, homopolymers of vinyl caprolactam, homopolymers of vinyl pyrrolidone or homopolymers of N-vinylformamide, copolymers of vinyl pyrrolidone and vinyl acetate, terpolymers of vinyl pyrrolidone, vinyl acetate and vinyl propionate, polyacrylamides, polyvinyl alcohols and polyethylene glycol/polypropylene glycol copolymers are preferred. Polyvinyl pyrrolidone and copolymers of vinyl pyrrolidone and nonionic comonomers, e.g. polyvinylpyrrolidone/vinyl acetate copolymers, are especially preferred. Suitable natural film-forming polymers include, e.g., hydroxyalkylated polysaccharides, especially hydroxyalkyl cellulose or hydroxyalkyl guar with preferably 2 or 3 carbon atoms in the alkyl groups.

(G) Special Embodiments Including Dye Compounds

Special embodiments of the products.-according to the invention are hair coloring or hair dyeing products, especially coloring fixing compositions. These compositions contain at least one hair coloring or dyeing substance. This can be a soluble organic dyestuff, especially a so-called direct dye compound or an inorganic or organic pigment.

The total amount of dyestuffs or dye precursor compounds in the composition of the invention is preferably about 0.01 to 7 percent by weight, especially preferably from about 0.2 to 4 percent by weight. Suitable direct-dyeing dye compounds include e.g. triphenylmethane dye compounds, aromatic nitro dye compounds, azo dye compounds, quinone dye compounds, cationic or anionic dye compounds. The following compounds are suitable:

Nitro-Dye Compounds (blue):

1,4-bis[(2-hydroxyethyl)amino]-2-nitrobenzene, 1-(2-Hydroxyethyl)amino-2-nitro-4-[di(2-hydroxyethyl)amino]-benzene (HC Blue No. 2), 1-Amino-3-methyl-4-[(2-hydroxyethyl)amino]-6-nitrobenzene (HC Violet No. 1), 4-[Ethyl-(2-hydroxyethyl)-amino]-1-[(2-hydroxyethyl)amino]-2-nitrobenzene hydrochloride(HC Blue No. 12), 4-[Di(2-hydroxyethyl)amino]-1-[(2-methoxyethyl)amino]-2-nitrobenzene (HC Blue No. 11), 1-[(2,3-Dihydroxypropyl)amino]-4-[methyl-(2-hydroxyethyl)amino]-2-nitrobenzene (HC Blue No. 10), 1-[(2,3-Dihydroxypropyl)-amino]-4-[ethyl-(2-hydroxyethyl)amino]-2-nitrobenzene hydrochloride(HC Blue No. 9), 1-(3-Hydroxypropylamino)-4-[di(2 hydroxyethyl)amino]-2-nitrobenzene (HC Violet No. 2), 1-Methylamino-4-[methyl-(2,3-dihydroxypropyl)amino]-2-nitrobenzene (HC Blue No. 6), 2-((4-Amino-2-nitrophenyl)amino)-5-dimethylaminobenzoic acid (HC Blue No. 13), 1-(2-Aminoethylamino)-4-[di(2-hydroxyethyl)amino]-2-nitrobenzene, 4-(Di(2-hydroxyethyl)amino)-2-nitro-1 -phenylaminobenzene.

Nitro Dye Compounds (Red):

1-Amino-4-[(2-hydroxyethyl)amino]-2-nitrobenzene (HC Red No. 7), 2-Amino-4,6-dinitrophenol, 1,4-Diamino-2-nitrobenzene (Cl76070), 4-Amino-2-nitro-diphenylamine (HC Red No. 1), 1-Amino-4-[di(2-hydroxyethyl)amino]-2-nitrobenzene hydrochloride(HC Red No. 13), 1 -Amino-5-chloro-4-[(2-hydroxyethyl)-amino]-2-nitrobenzene, 4-Amino-1-[(2-hydroxyethyl)amino]-2-nitrobenzene (HC Red No. 3), 4-((2-Hydroxyethyl)methylamino)-1-(methylamino)-2-nitrobenzene, 1-Amino-4-((2,3-dihydroxypropyl)amino)-5-methyl-2-nitrobenzene, 1-Amino-4-(methylamino)-2-nitrobenzene, 4-Amino-2-nitro-1-((prop-2-en-1-yl)-amino)benzene, 4-Amino-3-nitrophenol, 4-[(2-Hydroxyethyl)-amino]-3-nitrophenol, 4-[(2-Nitrophenyl)amino]phenol (HC Orange No. 1), 1-((2-Aminoethyl)-amino]-4-(2-hydroxyethoxy)-2-nitrobenzene (HC Orange No. 2), 4-(2,3-Dihydroxypropoxy)-1-[(2-hydroxyethyl)amino]-2-nitrobenzene (HC Orange No. 3); 1-Amino-5-chloro-4-[(2,3-dihydroxypropyl)amino]-2-nitrobenzene (HC Red No. 10), 5-Chlor-1,4-[di(2,3-dihydroxypropyl)amino]-2-nitrobenzene (HC Red No. 11), 2-[(2-Hydroxyethyl)-amino]-4,6-dinitrophenol, 4-Ethylamino-3-nitrobenzoic acid, 2-[(4-Amino-2-nitrophenyl)amino]benzoic acid, 2-Chlor-6-ethylamino-4-nitrophenol, 2-Amino-6-chloro-4-nitrophenol, 4-[(3-Hydroxypropyl)amino]-3-nitrophenol, 2,5-Diamino-6-nitropyridine, 6-Amino-3-((2-hydroxy-ethyl)amino)-2-nitropyridine, 3-Amino-6-((2-hydroxyethyl)amino)-2-nitropyridine, 3-Amino-6-(ethylamino)-2-nitropyridine, 3-((2-Hydroxyethyl)amino)-6-(methylamino)-2-nitropyridine, 3-Amino-6-(methylamino)-2-nitropyridine , 6-(Ethylamino)-3-((2-hydroxyethyl)amino)-2-nitropyridine, 1,2,3,4-Tetrahydro-6-nitroquinoxaline, 7-Amino-3,4-dihydro-6-nitro-2H-1,4-benzoxazine (HC Red No. 14).

Nitro Dye Compounds (Yellow):

1,2-Diamino-4-nitrobenzene (Cl76020), 1-Amino-2-[(2-hydroxyethyl)amino]-5-nitrobenzene (HC Yellow No. 5), 1-(2-Hydroxy-ethoxy)-2-[(2-hydroxyethyl)-amino]-5-nitrobenzene (HC Yellow No. 4), 1-[(2-Hydroxyethyl)amino]-2-nitrobenzene (HC Yellow No. 2), 2-(Di(2-hydroxyethyl)amino)-5-nitrophenol, 2-[(2-Hydroxyethyl)-amino]-1-methoxy-5-nitrobenzene, 2-Amino-3-nitrophenol, 1-Amino-2-methyl-6-nitrobenzene, 1-(2-Hydroxyethoxy)-3-methylamino-4-nitrobenzene, 2,3-(Dihydroxypropoxy)-3-methylamino-4-nitrobenzene, 2-[(2-Hydroxyethyl)amino]-5-nitrophenol (HC Yellow No. 11), 3-[(2-Aminoethyl)amino]-1-methoxy-4-nitrobenzene hydrochloride(HC Yellow No.9), 1-[(2-Ureidoethyl)-amino]-4-nitrobenzene, 4-[(2,3-Dihydroxypropyl)amino]-3-nitro-1-trifluormethyl-benzene (HC Yellow No. 6), 1-Chloro-2,4-bis[(2-hydroxyethyl)amino]-5-nitrobenzene (HC Yellow No. 10), 1-Amino-4-((2-aminoethyl)amino)-5-methyl-2-nitrobenzene, 4-[(2-Hydroxyethyl)-amino]-3-nitro-1 -methylbenzene, 1 -Chlor-4-[(2-hydroxyethyl)amino]-3-nitrobenzene (HC Yellow No. 12), 4-[(2-hydroxy-ethyl)amino]-3-nitro-1-trifluormethylbenzene (HC Yellow No. 13), 4-[(2-Hydroxyethyl)amino]-3-nitrobenzonitrile (HC Yellow No. 14), 4-[(2-Hydroxyethyl)amino]-3-nitro-benzamid (HC Yellow No. 15), 3-((2-Hydroxyethyl)amino)-4-methyl-1-nitrobenzene, 4-Chlor-3-((2-hydroxyethyl)-amino)-1-nitrobenzene.

Quinone Dye Compounds:

1,4-Di[(2,3-dihydroxypropyl)amino]-9,10-anthraquinone, 1,4-Di[(2-hydroxyethyl)-amino]-9,10-anthraquinone (Cl61545 Disperse Blue 23), 1-[(2-Hydroxyethyl)-amino]-4-methylamino-9,10-anthraquinone (Cl61505, Disperse Blue No. 3), 2-[(2-Aminoethyl)amino]-9,10-anthraquinone (HC Orange No. 5), 1-Amino-4-hydroxy-9,10-anthraquinone (Cl 60710, Disperse Red 15), 1-Hydroxy-4-[(4-methyl-2-sulfophenyl)amino]-9,10-anthraquinone, 7-Beta-D-glucopyranosyl-9,10-dihydro-1-methyl-9,10-dioxo-3,5,6,8-tetrahydroxy-2-anthracene carboxylic acid (Cl75470, Natural Red 4), 1-[(3-Aminopropyl)amino]-4-methylamino-9,10-anthraquinone (HC Blue No. 8), 1-((3-Aminopropyl)amino]-9,10-anthraquinone (HC Red No. 8), 1,4-Diamino-2-methoxy-9,10-anthraquinone (Cl62015, Disperse Red No. 11, Solvent Violet No. 26), 1,4-Dihydroxy-5,8-bis[(2-hydroxyethyl)-amino]-9,10-anthraquinone (Cl62500, Disperse Blue No. 7, Solvent Blue No. 69), 1,4-Diamino-9,10-anthraquinone (Cl61100, Disperse Violet No. 1), 1-Amino-4-(methylamino)-9,10-anthraquinone (Cl61105, Disperse Violet No. 4, Solvent Violet No. 12), 2-Hydroxy-3-methoxy-1,4-naphthaquinone, 2,5-Dihydroxy-1,4-naphthaquinone, 2-Hydroxy-3-methyl-1,4-naphthaquinone, N-(6-((3-Chloro-4-(methylamino)phenyl)imino)-4-methyl-3-oxo-1,4-cyclohexadien-1-yl)urea (HC Red No. 9), 2-((4-(Di(2-hydroxyethyl)amino)phenyl)amino)-5-((2-hydroxyethyl)-amino)-2,5-cyclohexadien-1,4-dione (HC Green No. 1), 5-Hydroxy-1,4-naphtha-quinone (Cl75500, Natural Brown No. 7), 2-Hydroxy-1,4-naphthoquinone (Cl75480, Natural Orange No. 6), 1,2-Dihydro-2-(1,3-dihydro-3-oxo-2H-indol-2-yliden)-3H-indol-3-one (Cl73000), 4-((5-((2-Hydroxyethyl)amino-1-methyl-1H-pyrazol-4-yl)imino)-4,5-dihydro-5-((2-hydroxyethyl)-imino)-1-methyl-1H-pyrazole sulfate (1:1), hydrate (1:1).

Basic Dye Compounds:

9-(Dimethylamino)-benzo[a]phenoxazin-7-ium chloride (Cl51175; Basic Blue No.6), Di[4-(diethylamino)phenyl][4-(ethylamino)naphthyl]carbenium chloride (Cl42595; Basic Blue No. 7), Di-(4-(dimethylamino)phenyl)-(4-(methylphenyl-amino)-napthalene-1-yl)carbenium chloride(Cl42563; Basic Blue No. 8), 3,7-Di(dimethylamino)phenothiazin-5-ium chloride (Cl52015 Basic Blue No. 9), Di[4-(dimethylamino)phenyl][4-(phenylamino)naphthyl]carbenium chloride (Cl44045; Basic Blue No. 26), 2-[(4-(Ethyl(2-hydroxyethyl)amino)phenyl)azo]-6-methoxy-3-methyl-benzothiazolium methyl sulfate (Cl11154; Basic Blue No. 41), 8-Amino-2-bromo-5-hydroxy-4-imino-6-[(3-(trimethylammonio)-phenyl)-amino]-1(4H)-napthalinone chloride(Cl56059; Basic Blue No. 99), bis-[4-(dimethylamino)phenyl]-[4-(methylamino)phenyl]carbenium chloride (Cl42535; Basic Violet No. 1), Tri(4-amino-3-methylphenyl)carbenium chloride (Cl42520; Basic Violet No. 2), Tris[4-(dimethylamino)-phenyl]carbenium chloride (Cl42555; Basic Violet No. 3), 2-[3,6-(Diethylamino)dibenzopyranium-9-yl]-benzoic acid chloride(C145170; Basic Violet No. 10), Di(4-aminophenyl)(4-amino-3-methyl-phenyl)carbenium chloride(Cl42510 Basic Violet No. 14), 1,3-bis[(2,4-diamino-5-methylphenyl)azo]-3-methylbenzene (Cl21010; Basic Brown No. 4), 1-[(4-Aminophenyl)azo]-7-(trimethylammonio)-2-naphthol chloride (Cl12250; Basic Brown No. 16), 3-[(4-Amino-2,5-dimethoxyphenyl)azo]-N,N,N-trimethylbenzene aminium chloride (Cl112605, Basic Orange No. 69), 1-[(4-Amino-2-nitrophenyl)azo]-7-(trimethylammonio)-2-naphthol chloride, 1-[(4-Amino-3-nitro-phenyl)azo]-7-(trimethylammonio)-2-naphthol chloride(Cl12251; Basic Brown No. 17), 3,7-Diamino-2,8-dimethyl-5-phenylphenazinium chloride(Cl50240; Basic Red No. 2), 1,4-Dimethyl-5-[(4-(dimethylamino)phenyl)azo]-1,2,4-triazolium chloride (Cl11055; Basic Red No. 22), 2-Hydroxy-1-[(2-methoxyphenyl)azo]-7-(trimethylammonio)-napthalene chloride (Cl12245; Basic Red No. 76), 2-[2-((2,4-Dimethoxyphenyl)amino)ethenyl]-1,3,3-trimethyl-3H-indolium chloride (Cl48055; Basic Yellow No. 11), 3-Methyl-1-phenyl-4-[(3-(trimethylammonio)-phenyl)azo]-pyrazol-5-one chloride (Cl12719; Basic Yellow No. 57), Di[4-(dimethylamino)phenyl]-iminomethane hydrochloride (Cl41000; Basic Yellow No. 2), bis-[4-(diethylamino)-phenyl]phenyl carbenium hydrogen sulfate (1:1) (Cl42040; Basic Green No. 1), Di(4-(dimethylamino)phenyl)phenylmethanol (Cl42000; Basic Green No. 4), 1-(2-Morpholinium-propylamino)-4-hydroxy-9,10-anthraquinone methyl sulfate, 1-((3-(Dimethylpropylaminium)propyl)amino]-4-(methylamino)-9,10-anthraquinone chloride.

Neutral Azo Dye Compounds:

1-[Di(2-hydroxyethyl)amino]-3-methyl-4-[(4-nitrophenyl)azo]-benzene (Cl11210, Disperse Red No. 17),1 -[Di(2-hydroxyethyl)amino]-4-[(4-nitrophenyl)azo]-benzene (Disperse Black No. 9), 4-[(4-Aminophenyl)azo]-1-[di(2-hydroxyethyl)-amino]-3-methylbenzene (HC Yellow No. 7), 2,6-Diamino-3-[(pyridine-3-yl)azo]-pyridine, 2-((4-(Acetylamino)phenyl)azo)-4-methylphenol (Cl11855; Disperse Yellow No. 3).

Acid Dye Compounds:

6-Hydroxy-5-[(4-sulfophenyl)azo]-2-napthalene sulfonic acid disodium salt (Cl15985; Food Yellow No. 3; FD&C Yellow No. 6), 2,4-Dinitro-1-naphthol-7-sulfonic acid disodium salt(Cl10316; Acid Yellow No. 1; Food Yellow No. 1), 2-(Indan-1,3-dion-2-yl)quinolin-x,x-sulfonic acid (mixture of mono and disulfonic acids) (Cl47005; D&C Yellow No. 10; Food Yellow No. 13; Acid Yellow No. 3), 5-Hydroxy-1-(4-sulfophenyl)-4-[(4-sulfophenyl)azo]pyrazol-3-carboxylic acid trisodium salt (Cl19140; Food Yellow No. 4; Acid Yellow No. 23), 9-(2-Carboxyphenyl)-6-hydroxy-3H-xanthen-3-one (Cl45350; Acid Yellow No. 73; D&C Yellow No. 8), 4-((4-Amino-3-sulfophenyl)azo)benzenesulfonic acid disodium salt (Cl13015, Acid Yellow No. 9), 5-[(2,4-Dinitrophenyl)amino]-2-phenylamino-benzene sulfonic acid sodium salt (Cl10385; Acid Orange No. 3), 4-[(2,4-Dihydroxyphenyl)azo] benzene sulfonic acid monosodium salt (Cl14270; Acid Orange No. 6), 4-[(2-Hydroxynaphth-1-yl)azo]-benzene sulfonic acid sodium salt (Cl15510; Acid Orange No. 7), 4-[(2,4-Dihydroxy-3-[(2,4-dimethylphenyl)azo]-phenyl)azo]-benzene sulfonic acid sodium salt (Cl20170; Acid Orange No. 24), 4-Hydroxy-3-[(4-sulfonaphth-1-yl)azo]-1-napthalene sulfonic acid disodium salt (Cl14720; Acid Red No. 14),.4-Hydroxy-3-[(2-methoxyphenyl)azo]-1-napthalene sulfonic acid monosodium salt (Cl14710; Acid Red No. 4), 6-Hydroxy-5-[(4-sulfo-naphth-1-yl)azol-2,4-napthalene-disulfonic acid trisodium salt (Cl16255; Ponceau 4R; Acid Red No. 18), 3-Hydroxy-4-[(4-sulfonaphth-1-yl)azo]-2,7-napthalene-disulfonic acid trisodium salt (Cl16185; Acid Red No. 27), 8-Amino-1-hydroxy-2-(phenyl-azo)-3,6-napthalene-disulfonic acid disodium salt (Cl17200; Acid Red No. 33), 5-(acetylamino)-4-hydroxy-3-[(2-methylphenyl)azo]-2,7-napthalene disulfonic acid disodium salt(Cl18065; Acid Red No. 35), 2-(3-Hydroxy-2,4,5,7-tetraiodo-dibenzopyran-6-on-9-yl)-benzoic acid disodium salt (Cl45430; Acid Red No. 51), N-[6-(Diethylamino)-9-(2,4-disulfophenyl)-3H-xanthen-3-yliden]-N-ethyl ethane ammonium hydroxide, inner salt, sodium salt (Cl45100; Acid Red No. 52), 8-[(4-(Phenylazo)phenyl)azo]-7-naphthol-1,3-disulfonic acid disodium salt (Cl27290; Acid Red No. 73), 2′,4′,5′,7′-Tetrabrom-3′,6′-dihydroxyspiro[iso-benzofuran-1(3H),9′-[9H]xanthen]-3-one disodium salt (Cl45380 Acid Red No. 87), 2′,4′,5′,7′-Tetrabromo-4,5,6,7-tetrachloro -3′,6′-dihydroxyspiro-[isobenzofuran-1(3H),9′[9H]xanthen]-3-one disodium salt (Cl145410; Acid Red No. 92), 3′,6′-Dihydroxy-4′,5′-diiodospiro[isobenzofuran-1(3H),91 (9H)-xanthen]-3-one disodium salt (Cl45425; Acid Red No. 95), 2-Hydroxy-3-((2-hydroxy-naphth-1-yl)azo)-5-nitrobenzene sulfonic acid monosodium salt (Cl15685; Acid Red No. 184), (2-Sulfophenyl)-di[4-(ethyl((4-sulfophenyl)methyl)amino)phenyl]-carbenium, disodium salt, betaine (Cl42090; Acid Blue No. 9; FD&C Blue No. 1), 1,4-bis-[(2-sulfo-4-methylphenyl)amino]-9,10-anthraquinone-disodium salt (Cl 61570; Acid Green No. 25), bis[4-(dimethylamino)phenyl]-(3,7-disulfo-2-hydroxynaphth-1-yl)carbenium inner salt, monosodium salt (Cl44090; Food Green No. 4; Acid Green No. 50), bis-[4-(diethylamino)-phenyl](2,4-disulfo-phenyl)carbenium inner salt, sodium salt (2:1) (Cl42045; Food Blue No. 3; Acid Blue No. 1), bis[4-(diethylamino)phenyl](5-hydroxy-2,4-disulfophenyl)-carbenium inner salt, calcium salt (2:1) (Cl42051; Acid Blue No. 3), 1-Amino-4-(cyclo-hexylamino)-9,10-anthraquinone-2-sulfonic acid sodium salt (Cl62045; Acid Blue No. 62), 1-Amino-4-(phenylamino)-9,10-anthraquinone-2-sulfonic acid (Cl62055; Acid Blue No. 25), 2-(1,3-Dihydro-3-oxo-5-sulfo-2H-indol-2-yliden)-2,3-dihydro-3-oxo-1H-indol-5-sulfonic acid disodium salt (Cl73015; Acid Blue No. 74), 9-(2-carboxyphenyl)-3-[(2-methylphenyl)amino]-6-[(2-methyl-4-sulfophenyl)-amino]xanthylium inner salt, monosodium salt (Cl45190; Acid Violet No. 9), 1-Hydroxy-4-[(4-methyl-2-sulfophenyl )amino]-9,10-anthraquinone-sodium salt (Cl60730; D&C Violet No. 2; Acid Violet No. 43), bis[3-nitro-4-[(4-phenylamino)-3-sulfophenylamin)]-phenyl]sulfone (Cl10410; Acid Brown No. 13), 5-Amino-4-hydroxy-6-[(4-nitrophenyl)-azo]-3-(phenylazo)-2,7-napthalene disulfonic acid disodium salt (Cl20470; Acid Black No. 1), 3-Hydroxy-4-[(2-hydroxynaphth-1-yl)azo]-7-nitro-1-napthalene-sulfonic acid chromium complex (3:2) (Cl15711; Acid Black No. 52), 3-[(2,4-Dimethyl-5-sulfophenyl)azo]-4-hydroxy-1-napthalene sulfonic acid disodium salt (Cl14700; Food Red No. 1; Ponceau SX; FD&C Red No. 4), 4-(Acetylamino)-5-hydroxy-6-[(7-sulfo-4-[(4-sulfophenyl)azo]naphth-1-yl)azo]-1,7-napthalene disulfonic acid tetrasodium salt (Cl28440; Food Black No. 1), 3-Hydroxy-4-(3-methyl-5-oxo-1 -phenyl-4,5-dihydro-1H-pyrazol-4-yl-azo)-napthalene-1-sulfonic acid sodium salt, chromium complex (Acid Red No. 195).

E. Sagarin, “Cosmetics, Science and Technology”, Interscience Publishers Inc., New York (1957), pp. 503 ff.; H. Janistyn, “Handbook of Cosmetics and Fragrances”, Vol 3 (1973), pp. 388 ff. and K. Schrader “Foundations and Formulations of Cosmetics”, 2^{nd} Ed. (1989), pp. 782 to 815 describe additional or further known and conventional hair dye compounds for dyeing hair, which can be contained in the hair dye compositions according to the invention.

Suitable hair dying pigments are coloring agents, which are practically insoluble in the application medium, and can be inorganic or organic. Also inorganic-organic mixed pigments are possible. The pigments however are preferably not nanopigments. The preferred particle size amounts to 1 to 200 μm, especially 3 to 150 μm, and even more preferably from 10 to 100 μm. Inorganic pigments are preferred. The inorganic pigments can be of a natural origin, for example chalk, ocher, umber, green earth, burnt Terra di Siena or graphite. The pigments can be white pigments, such as titanium dioxide or zinc oxide, black pigments, such as iron oxide black, colored pigments, such as ultramarine or iron oxide red, lustrous pigments, metal effect pigments, pearlescent pigments and fluorescence or phosphorescence pigments. Preferably at least one pigment is a non-white pigment. Metal oxides, metal hydroxides and metal oxihydrates, mixed phase pigments, sulfur-containing silicates, metal sulfides, complex metal cyanides, metal sulfates, metal chromates and metal molybdates, as well as metals themselves (Bronze pigments) are suitable. Especially titanium dioxide (Cl 77891), black iron oxide (Cl 77499), yellow iron oxide (Cl 77492), red and brown iron oxide (Cl 77491), mangan violet (Cl 77742), Ultramarine (Sodium aluminum sulfo silicate, Cl 77007, Pigment Blue 29), Chromium oxide hydrate (Cl 77289), iron blue (Ferric ferrocyanide, Cl 77510), Carmine (Cochineal), are suitable as the pigments in embodiments of the cosmetic preparations according to the invention. Pigments based on mica, which are coated with a metal oxide or a metal oxychloride, such as titanium dioxide or bismuth oxychloride and, if 15 necessary,.other coloring agents, such as iron oxides, iron blue, Ultramarine, Carmine, etc, are particularly preferred. The colors of these particular preferred pigments are determined by variation of the coating thickness. These pigments are marketed by Merck, Germany, under the trademarks RONA®, COLORONA®, DICHRONA® and TIMIRON®. Organic pigments include, e.g., the natural pigments sepia, gamboge, animal charcoal, Kasseler brown, indigo, chlorophyll and other plant pigments. Synthetic organic pigments are, e.g., azo pigments, anthraquinoid, indigoid, dioxazine, quinacridone, phthalocyanine, isoindolinone, perylene and perinone, metal complex, alkali blue and diketopyrrolopyrrole pigments.

The product according to the invention can also contain conventional cosmetic additives usually used in hair treatment compositions, e.g. perfume oils in an amount of 0.01 to 0.5 percent by weight; preservatives, such as parabene, in an amount of 0.01 to 1.0percent by weight; buffer substances, such as sodium citrate or sodium phosphate, in an amount of 0.1 to 1.0 percent by weight; care substances, such as e.g. plant extracts and vegetable extracts, protein hydroylzates and silk hydrolyzates, lanolin derivatives, in an amount of 0.1 to 5 percent by weight; physiologically compatible silicone derivatives, such as volatile or non-volatile silicone oils or high molecular weight siloxane polymers in an amount of 0.05 to 20 percent by weight; light protective agents, antioxidants, radical-trapping agents, anti-flaking active ingredients, direct dye compounds, luster-imparting substances and combability-improving substances in an amount of 0.01 to 2 percent by weight.

The aerosol foam product according to the invention is filled in a pressure-resistant aerosol container or package, which is provided with a device for foaming production (aerosol foam head). When a foam is produced with this aerosol container or can, it is worked gently into the hair and rapidly breaks up when it is worked into the hair.

The method of using the cosmetic composition according to the invention comprises applying the composition to moist hand-towel dried hair in an amount sufficient to produce the desired hair-care or hair-fixing effect on the hair and is left on the hair without rinsing. Subsequently a hairstyle can be formed in the usual way or the hair can be set or curled and styled finally dry. However it is also possible to use the composition directly on dried hair.

Foam with good strand drawing or forming properties and good compact foam quality is obtained, which breaks up quickly in the hair and is easily worked into the hair. It is characterized by good styling properties, i.e. by an especially good easy hair brushing during dry styling of moist treated hair, pleasant smooth feel of the moist and dry hair, good elasticity, no loading of the hair, beautiful luster and strong fixing.

### BRIEF DESCRIPTION OF THE DRAWING

The objects, features and advantages of the invention will now be illustrated in more detail with the aid of the following description of the preferred embodiments, with reference to the accompanying sole FIGURE, which is a photographic view of the strand drawing properties of a foam composition according to Example 1A.

The following examples should illustrate the subject matter of the invention in more detail, without limiting the claims appended below.

### EXAMPLES

The subsequently described effective ingredient mixture was filled into an aerosol can with a foam valve with propane/butane (4.8 bar) as propellant in a ratio of 92:8. The described foam properties relate to fresh-made foam, immediately after being dispensed from the aerosol can.

The judging of the foam quality and application properties occurred in salon tests. The same amount of foam was applied to each tested individual. A panel of hairstyling experts judged the foam and the hair treated with the foam.

### Example 1

Tested Foamable Compositions

Tested compositions A, B and C are reported in Table I.

TABLE I
EFFECTIVE INGREDIENT MIXTURES FOR AEROSOL HAIR FOAM

| Ingredient | A (g) | B (g) | C (g) |
|---|---|---|---|
| (A) MERQUAT ® 550¹ | 6.9 | 13.4 | 2.27 |
| (B) LUVIFLEX ® SOFT² | 5.6 | 3.73 | 6.5 |
| Aminomethylpropanol | 0.9 | 0.6 | 1.0 |
| (95%) | | | |
| Laureth-4 | 0.2 | 0.2 | 0.2 |
| Cetyltrimethyl ammonium chloride | 0.15 | 0.15 | 0.15 |
| Ethanol | 10 | 10 | 10 |
| Water | To 100 | To 100 | To 100 |
| Solid ratio (A):(B) | 1:3 | 1:1 | 1:11 |

¹Polyquaternium-7, dimethyldiallyl ammonium chloride/acrylamide copolymer, 8% in water;
²Acrylates copolymer, ethylacrylate/methacrylic acid copolymer, 30% in water

Foam quality of Foam 1A: very compact, strand forming

Foam quality of Foam 1B: little strand forming, slimy, greasy

Application properties of Foam 1A: smooth feel in moist hair, good moist combability, good styling properties during hair styling, good shaping properties during curl styling, already definite curl formation during curl styling, setting phase during drying, beautiful smooth feel, does not load the hair, good elasticity and very beautiful luster.

Foam 1C: little strand forming or drawing, poor combability, little elasticity in dry hair, little definition of the hair, weak fixing, otherwise about the same as 1s foam 1A.

### Example 2

Comparative Compositions

Table II describes the ingredients and amounts of the comparative compositions.

TABLE II
COMPARATIVE FOAMABLE COMPOSITIONS

| Ingredient | A (g) | B (g) | C (g) | D (g) |
|---|---|---|---|---|
| (A′) CELQUAT ® L200¹ | 0.55 | 1.1 | — | — |
| (A′) LUVIQUAT ® HM 522² | — | — | 2.75 | 5.6 |
| (B) LUVIFLEX ® SOFT | 5.6 | 3.73 | 5.6 | 3.73 |
| Aminomethylpropanol | 0.9 | 0.6 | 0.9 | 0.6 |
| (95%) | | | | |
| Ethanol | 10 | 10 | 10 | 10 |
| Laureth-4 | 0.2 | 0.2 | 0.2 | 0.2 |
| Cetyltrimethyl ammonium | 0.15 | 0.15 | 0.15 | 0.15 |
| chloride | | | | |
| Water | To 100 | To 100 | To 100 | To 100 |
| Solid ratio (A′):(B) | 1:3 | 1:1 | 1:3 | 1:1 |

¹Polyquaternium-4, dimethyldiallyl ammonium chloride/hydroxy-ethylcellulose copolymer;
²Polyquaternium-16, vinylimidazolium methochloride/vinylpyrrolidone copolymer, 20% in water.

Comparing foam 2A and 2B to foam 1A: Poorer styling properties in comparison to the invention, more difficult setting of the hair, dull and lusterless appearance of the hair and a higher load on the hair.

Comparing foam 2C and 2D to foam 1A: very poor styling properties in comparison to the invention, the hair appears dry and dull, sticky and unpleasant loading of the hair.

### Example 3

Aerosol Foam-fixing Composition

| | | |
|---|---|---|
| 6.9 | g | MERQUAT ® 550 |
| 5.6 | g | LUVIFLEX ® SOFT |
| 0.9 | g | aminomethylpropanol (95%) |
| 1.5 | g | Vinylpyrrolidone/vinyl acetate Copolymer |
| | | (LUVISKOL ® VA64) |
| 0.2 | g | Laureth-4 |
| 0.3 | g | Perfume |
| 0.15 | g | Cetyltrimethylammonium chloride |
| 10 | g | ethanol |
| to 100 | g | Water |

The effective ingredient mixture is filled into an aerosol can with a foam valve together with propane/butane, 4.8 bar, as propellant, in a ratio 92:8.

### Example 4

Foam-fixing and Dyeing Composition

| | | |
|---|---|---|
| 6.9 | g | MERQUAT ® 550 |
| 5.6 | g | LUVIFLEX ® SOFT |
| 0.9 | g | aminomethylpropanol (95%) |
| 0.2 | g | Laureth-4 |
| 0.2 | g | Perfume |
| 0.11 | g | 3-[(2′-nitro-4′-(trifluoromethyl)phenyl)aminol-1,2- |
| | | propandiol |
| 0.15 | g | Cetyltrimethylammonium chloride |
| 10 | g | Ethanol |
| to 100 | g | Water |

The effective ingredient mixture is filled into an aerosol can with a foam valve together with propane/butane, 5 bar, as propellant, in a ratio 94:6.

### Example 5

Coloring Mousse Red

| | | |
|---|---|---|
| 5.00 | g | TIMIRON ® STARLIGHT RED (Merck)* |
| 6.9 | g | MERQUAT ® 550 |
| 5.6 | g | LUVIFLEX ® SOFT |
| 0.9 | g | aminomethylpropanol (95%) |
| 0.2 | g | Laureth-4 |
| 0.2 | g | Perfume |
| 0.15 | g | Cetyltrimethylammonium chloride |
| 10 | g | Ethanol |
| to 100 | g | Water |

*Mica/titanium dioxide pigment with red reflection color

The effective ingredient mixture is filled into an aerosol can with a foam valve together with propane/butane, 5 bar, as propellant, in a ratio 94:6.

The disclosure in German Patent Application 102 28 436.9 of Jun. 26, 2002 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in an aerosol foam product for hair treatment, it is not intended to be limited to the

While/the invention has been illustrated and described as embodied in an aerosol foam product for hair treatment, it is not intended to be limited to the details shown, since various modifications and changes may be made without departing in any way from the spirit of the present invention.

Without further analysis, the foregoing will so fully reveal the gist of the present invention that others can, by applying current knowledge; readily adapt it for various applications without omitting features that, from the standpoint of prior art, fairly constitute essential characteristics of the generic or specific aspects of this invention.

What is claimed is new and is set forth in the following appended claims.

We claim:

1. An aerosol foam product for hair treatment, comprising
a pressure-resistant container;
a foamable composition containing at least one dialkyldiallyl ammonium chloride/acrylamide copolymer; at least one other copolymer, said at least one other copolymer comprising at least one first monomer and at least one second monomer, said at least one first monomer consisting of an alkyl acrylate and/or an alkyl methacrylate and said at least one second monomer consisting of acrylic acid and/or methacrylic acid; at least one base for at least partial or total neutralization of the at least one other copolymer and a solvent system; and
at least one propellant, said at least one propellant being filled together with said foamable composition in said pressure-resistant container.

2. The aerosol foam product as defined in claim 1, wherein said at least one dialkyldiallyl ammonium chloride/acrylamide copolymer consists of a dimethyldiallyl ammonium chloride/acrylamide copolymer.

3. The aerosol foam product as defined in claim 1, wherein said at least one dialkyldiallyl ammonium chloride/acrylamide copolymer has a cationic charge density from 200 to 250, expressed as molecular weight per unit cationic charge.

4. The aerosol foam product as defined in claim 1, wherein said at least one other copolymer is an alkylacrylate/methacrylic acid copolymer and alkyl groups of said alkylacrylate/methacrylic acid copolymer have from 1 to 4 carbon atoms.

5. The aerosol foam product as defined in claim 1, wherein said at least one other copolymer has a negative charge, said at least one dialkyldiallyl ammonium chloride/acrylamide copolymer has a positive charge and a ratio of said negative charge to said positive charge is in a range of 1:1 to 10:1.

6. The aerosol foam product as defined in claim 1, wherein foam formed from said foamable composition has strand drawing or forming properties.

7. The aerosol foam product as defined in claim 1, wherein said at least one base is an aminoalkylalcohol.

8. The aerosol foam product as defined in claim 1, wherein said foamable composition includes at least one surfactant.

9. The aerosol foam product as defined in claim 8, wherein said at least one surfactant is at least one of a nonionic surfactant and a cationic surfactant, and
wherein said nonionic surfactant is selected from the group consisting of fatty acid glyceride ethoxylates, fatty alcohol ethoxylates, fatty amine ethoxylates, fatty acid alkanol amide ethoxylates, fatty acid ester ethoxylates, ethoxylated fatty acid sugar esters, ethoxylated sorbitan fatty acid esters, non-ethoxylated fatty acid sugar esters and alkylpolyglycosides; and
wherein said cationic surfactant is of formula (I):
N⁽⁺⁾R¹R²R³R⁴X⁽⁻⁾ (I),
wherein R¹, R², R³ and R⁴, independently of each other, each represent an alkyl group, an aryl group, an alkoxy group, a polyoxyalkylene group, an alkylamido group, a hydroxyalkyl group or an alkyaryl group, each group with 1 to 22 carbon atoms, and X⁽⁻⁾ represents an anion.

10. The aerosol foam product as defined in claim 1, wherein said at least one propellant is propane, i-butane, n-butane or dimethyl ether or a mixture thereof.

11. The aerosol foam product as defined in claim 8, containing from 0.1 to 10 percent by weight of said at least one dialkyldiallyl ammonium chloride/acrylamide copolymer, from 0.1 to −10 percent by weight of said at least one other copolymer, an amount of said at least one base sufficient to neutralize from 50 to 100% of said at least one other copolymer, from 1 to 20 percent by weight of said at least one propellant, from 50 to 98 percent by weight of said solvent system and from 0 to 15 percent by weight of said at least one surfactant.

12. The aerosol foam product as defined in claim 1, wherein said composition comprises at least one nonionic film-forming polymer.

13. The aerosol foam product as defined in claim 12, wherein said at least one nonionic film-forming polymer is selected from the group consisting of polyvinyl pyrrolidone and copolymers of vinyl pyrrolidone and ethylenically unsaturated nonionic comonomers.

14. The aerosol foam product as defined in claim 1, wherein said composition contains at least one hair-dyeing substance.

15. A aerosol foam product for treating hair, said product comprising a composition for foam production and said composition comprising
from 0.1 to 5 percent by weight of dimethyldiallyl ammonium chloride/acrylamide copolymer;
from 0.1 to 5 percent by weight of ethylacrylate/methacrylic acid copolymer;
a base in an amount sufficient for neutralizing from 50 to 100 percent of said ethylacrylate/methacrylic acid copolymer;
from 50 to 98 percent by weight of a solvent, said solvent containing water, C₂-C₃ alcohol or mixtures thereof; and
from 1 to 20 percent by weight of a C₃- to C₄-hydrocarbon as propellant; wherein the ethylacrylate/methacrylic acid copolymer and the dimethyldiallyl ammonium chloride/acrylamide copolymer are present in a weight ratio of 1:1 to less than 10:1.

16. An aerosol foam product for treating hair, said product comprising a pressure-resistant container and a composition for foam production filled in the pressure-resistant container, and wherein said composition contains at least one hair-fixing polymer, at least one propellant and at least one solvent system, and wherein foam formed from said composition has strand drawing or forming properties after being dispensed from said pressure-resistant container.

17. A method of treating hair or of making curled or waved hair from said hair, said method comprising the steps of:
a) providing a foamable composition together with an aerosol propellant in a pressure-resistant container, said foamable composition comprising at least one dialkyldiallyl ammonium chloride/acrylamide copolymer; at least one other copolymer, said at least one other copolymer comprising at least one first monomer and at least one second monomer, said at least one first monomer consisting of an alkyl acrylate and/or an alkyl methacrylate and said at least one second monomer consisting of acrylic acid and/or methacrylic acid; at least one base for at least partial or total neutralization of the at least one other copolymer and a solvent system;
b) dispensing foam from said pressure-resistant container; and
c) applying said foam to said hair.
